(19) United States
(12) Patent Application Publication
(10) Pub. No.: US 2022/0251111 A1
(43) Pub. Date: Aug. 11, 2022

(54) PROCESS FOR THE PREPARATION OF AMORPHOUS MIDOSTAURIN WITH A LOW CONTENT OF RESIDUAL ORGANIC SOLVENT

(71) Applicant: Procos S.P.A., Cameri (NO) (IT)

(72) Inventors: Alberto Lena, Galliate (NO) (IT); Matteo Baudino, Rivarolo Canavese (TO) (IT); Gabriele Prina Cerai, Veglio (BI) (IT); Antonio Toppino, Monta' (CN) (IT); Jacopo Roletto, Torino (IT); Paolo Paissoni, Druento (TO) (IT)

(73) Assignee: Procos S.P.A., Cameri (NO) (IT)

(21) Appl. No.: 17/628,578

(22) PCT Filed: Aug. 5, 2020
(86) PCT No.: PCT/IB2020/057404

§ 371 Date: Jan. 20, 2022

(30) Foreign Application Priority Data
Aug. 8, 2019 (IT) 10 2019 000014346

(51) Int. Cl. C07D 498/22

(52) U.S. Cl. CPC C07D 498/22; C07B 2200/13

(57) ABSTRACT

The present invention relates to a process for the preparation of an amorphous form of midostaurin with a low content of residual organic solvent.

### FIELD OF THE INVENTION

The present invention relates to a process for the preparation of an amorphous form of midostaurin with a low content of residual organic solvent.

### STATE OF THE ART

Midostaurin is an antitumor active pharmaceutical ingredient approved by the FDA and EMA in 2017 for the treatment of adults with acute myeloid leukemia (AML) and for the treatment of adults with aggressive systemic mastocytosis, systemic mastocytosis associated with a hematologic malignancy, and mastocytic leukemia. The active ingredient midostaurin (INN), also known as N-benzoyl-Staurosporine or with the abbreviation PKC-412, has the following formula (I)

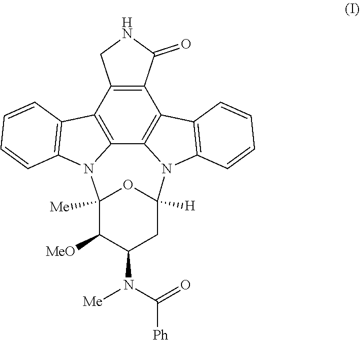 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

and is marketed as a medicinal product in capsules for oral administration under the name Rydapt®.

Despite the recent approval as an antitumor medicine, midostaurin has been a compound known for several decades and is prepared by semisynthesis from Staurosporin, an alkaloid produced by fermentation from the bacterium Streptomyces staurosporeus, through a benzoylation reaction (see, for example, U.S. Pat. No. 5,093,330 and JPH05247055).

However, the Applicant has found that, although there is a large literature on midostaurin and the use thereof as an antineoplastic drug, there are few examples in the literature and on the market of solid forms of said active ingredient, although the solid form has numerous advantages in terms of handling, formulation and storage.

In U.S. Pat. No. 5,093,330 and JPH05247055 the synthesis of midostaurin and the isolation thereof respectively through chromatographic purification followed by removal of the solvent or dispersion and subsequent filtration in isopropyl ether is reported.

The Applicant has noted that said purification is particularly complicated and expensive.

WO200648296A1 describes the preparation of the amorphous form of midostaurin through the use of the spray drying technique using dichloromethane or tetrahydrofuran as dissolution solvents or alternatively an ethanol/acetic acid mixture in the proportion 70:30.

The Applicant has noted that the spray drying technique requires the use of expensive equipment and generally involves consistent losses of powdery product due to the flow of inert gas in countercurrent with respect to the jet of the liquid solution containing the product subject to amorphization. The Applicant finds that the product losses occurring during the preparation phases of the active ingredient entail an increase in production costs, with consequent losses in efficiency and competitiveness of the process itself.

WO2018165071 describes a new procedure for isolating amorphous midostaurin by precipitation from a solution of dimethylformamide with heptane or with water. The Applicant has found that this procedure does not however allow to obtain a product in which the values of the residual solvents fall within the limits prescribed by the International Council for Harmonization of Technical Requirements for Pharmaceuticals for Human Use (ICH) and that therefore they do not allow, in the absence of further expensive purification steps, the use in the pharmaceutical field.

### SUMMARY OF THE INVENTION

Therefore, the object of the present invention is to develop a simple, effective, competitive, economical and industrially applicable method for the preparation of an amorphous form of midostaurin with a content of residual organic solvent falling within the limits prescribed by the International Council for Harmonization of Technical Requirements for Pharmaceuticals for Human Use (ICH) and which therefore allows the use thereof in the pharmaceutical field.

The Applicant has surprisingly observed that it is possible to prepare an amorphous form of midostaurin with a content of residual organic solvent within the limits prescribed by the ICH by precipitation from a solution composed of DMSO and water followed by washing of the filtered product with water and subsequent drying.

In a first aspect thereof, therefore, the present invention relates to a process for the preparation of an amorphous form of midostaurin, comprising the steps of:

a) preparing a solution of midostaurin in dimethylsulfoxide;

b) combining the solution of step a) with a first amount of water so as to obtain a first suspension of midostaurin;

c) filtering the first suspension of step b), obtaining a first filtered solid comprising midostaurin;

d) suspending under stirring the first filtered solid of step c) in a second amount of water so as to obtain a second suspension of midostaurin;

e) filtering the second suspension of step d), obtaining a second filtered solid comprising midostaurin; and

f) drying the second filtered solid of step e) so as to obtain a dried solid, wherein said dried solid is said amorphous form of midostaurin.

The process according to the present invention is very simple, effective and industrially applicable but above all economically very convenient with respect to the use of the spray drying technique. This technology, in fact, requires the implementation of a plant dedicated to drying by atomization which involves high investment costs, in addition to causing inevitable losses of material during processing.

Furthermore, the process according to the present invention allows to obtain an amorphous midostaurin with a low content of residual organic solvent, in such an amount so as to fall within the limits prescribed by the International Council for Harmonization of Technical Requirements for Pharmaceuticals for Human Use (ICH), which for the organic solvent used in the present process, dimethylsulfoxide, is 5000 ppm.

In a further aspect thereof, therefore, the present invention relates to an amorphous form of midostaurin comprising a residual amount of dimethylsulfoxide, wherein said residual amount of dimethylsulfoxide is less than or equal to 5000 ppm.

The Applicant has in fact found that said specific properties result from the process according to the present invention, from which the amorphous form of midostaurin according to the present invention is obtainable, and distinguish the latter from other known forms of midostaurin, also in the amorphous form.

In a preferred embodiment of said amorphous form of midostaurin, said residual amount of dimethylsulfoxide varies from 100 ppm to 5000 ppm, more preferably it varies from 150 ppm to 2500 ppm.

Thanks to the low content of residual organic solvent, which falls within the limits prescribed by the International Council for Harmonization of Technical Requirements for Pharmaceuticals for Human Use (ICH), it is possible to use the amorphous form of midostaurin according to the present invention in the pharmaceutical field, and in particularly for the treatment of tumors, in particular acute myeloid leukemia and mastocytosis, such as for example systemic mastocytosis, also in the aggressive systemic mastocytosis forms thereof, systemic mastocytosis associated with a hematologic malignancy, and mastocytic leukemia.

In a further aspect, the present invention therefore relates to a pharmaceutical composition comprising the amorphous form of midostaurin according to the present invention, and at least one component selected from the group consisting of: a pharmaceutically acceptable excipient, and a pharmaceutically acceptable solvent.

In a further aspect thereof, moreover, the present invention relates to the amorphous form of midostaurin according to the present invention for use as a medicament.

In a further aspect thereof, moreover, the present invention relates to the amorphous form of midostaurin according to the present invention for use in the treatment of a tumor.

In a preferred embodiment, said tumor is acute myeloid leukemia or mastocytosis, more preferably a systemic mastocytosis, even more preferably a systemic mastocytosis selected from the group consisting of: aggressive systemic mastocytosis, systemic mastocytosis associated with a hematologic malignancy, and mastocytic leukemia.

### BRIEF DESCRIPTION OF THE DRAWINGS

FIG. 1 shows the X-ray diffractogram of the amorphous form of midostaurin obtained in Example 1.

### DETAILED DESCRIPTION OF THE INVENTION

In a first aspect thereof, the present invention relates to a process for the preparation of an amorphous form of midostaurin, comprising the steps of:

a) preparing a solution of midostaurin in dimethylsulfoxide;

b) combining the solution of step a) with a first amount of water so as to obtain a first suspension of midostaurin;

c) filtering the first suspension of step b), obtaining a first filtered solid comprising midostaurin;

d) suspending under stirring the first filtered solid of step c) in a second amount of water so as to obtain a second suspension of midostaurin;

e) filtering the second suspension of step d), obtaining a second filtered solid comprising midostaurin; and

f) drying the second filtered solid of step e) so as to obtain a dried solid, wherein said dried solid is said amorphous form of midostaurin.

The process according to the present invention is very simple, effective and industrially applicable but above all economically very convenient with respect to the use of the spray drying technique. This technology, in fact, requires the implementation of a plant dedicated to drying by atomization which involves high investment costs, in addition to causing inevitable losses of material during processing.

Furthermore, the process according to the present invention allows to obtain an amorphous midostaurin with a low content of residual organic solvent, in such an amount so as to fall within the limits prescribed by the International Council for Harmonization of Technical Requirements for Pharmaceuticals for Human Use (ICH), which for the organic solvent used in the present process, dimethylsulfoxide, is 5000 ppm.

The present invention can have, in one or more of its aspects, one or more of the preferred characteristics set forth below, which can be combined as desired with each other according to the application requirements.

Within the context of the present description and following claims, all the numerical magnitudes indicating quantities, parameters, percentages, and so on are to be considered preceded in every circumstance by the term “about” unless indicated otherwise. In addition, all ranges of numerical quantities include all possible combinations of the maximum and minimum numerical values and all possible intermediate ranges, in addition to those specifically indicated below.

Furthermore, within the scope of the present description and in the subsequent claims, all the quantities expressed in, or referring to, units of volume, for example milliliters or “parts in volume”, are to be understood as defined at a temperature of 25° C.

The process according to the present invention comprises a step a) of preparing a solution of midostaurin in dimethylsulfoxide.

The midostaurin to be used for the provision of the solution of said step a), can be synthesized according to any of the methods known to the skilled in the art for this purpose, such as for example the synthesis reported in the Italian patent application 102019000004729.

Preferably, in said solution of step a) the amount of dimethylsulfoxide varies from 1 to milliliters per gram of midostaurin, more preferably it varies from 5 to 20 milliliters per gram of midostaurin, optimally said amount of dimethylsulfoxide being 10 milliliters per gram of midostaurin.

In step b) of the process according to the present invention, the solution of step a) is combined with a first amount of water, thus leading to the precipitation of the midostaurin in solution and so as to obtain a first suspension of midostaurin.

Preferably, in said step b) said first amount of water varies from 1 to 30 milliliters per gram of midostaurin in the solution of step a), more preferably it varies from 5 to 20 milliliters per gram of midostaurin in the solution of step a), optimally said first amount of water is 10 milliliters per gram of midostaurin in the solution of step a).

In a preferred embodiment of the process according to the present invention, said first amount of water varies from 0.5 to 1.5 parts in volume, more preferably it varies from 0.75 to 1.25 parts in volume, per part in volume of dimethylsulfoxide of the solution of step a). In a particularly preferred embodiment of the process according to the present invention, said first amount of water is 1 part in volume per part in volume of dimethylsulfoxide of the solution of step a).

Preferably, step b) of the process according to the present invention is carried out at a temperature varying from 20° C. to 40° C., more preferably varying from 20° C. to 30° C., optimally said temperature being 25° C.

In said step b) said operation of combining the solution of step a) with a first amount of water can be carried out either by adding said solution of step a) to said first amount of water, or vice versa, or by adding said first amount of water to said solution of step a).

In both cases, the addition time preferably varies from 5 minutes to 3 hours, more preferably it varies from 15 minutes to 1 hour, optimally said addition time being 30 minutes.

In an embodiment of the process according to the present invention, therefore, in said step b) said solution of step a) is added to said first amount of water in a time varying from 5 minutes to 3 hours, more preferably varying from 15 minutes to 1 hour, said addition time being 30 minutes.

In another embodiment of the process according to the present invention, therefore, in said step b) said first amount of water is added to said solution of midostaurin of step a) in a time varying from 5 minutes to 3 hours, more preferably it varies from 15 minutes to 1 hour, optimally said addition time being 30 minutes.

This last embodiment allows to speed up the subsequent step c) of the process according to the present invention, as a consequence of a better filterability of the obtained suspension.

Preferably, in said step b) of the process according to the present invention said operation of combining is carried out by mixing said solution of step a) and said first amount of water.

Preferably, in said step b) said first suspension of midostaurin is maintained under stirring after said solution of step a) has been combined with said first amount of water, for a time varying from 30 minutes to 3 hours, more preferably varying from 45 minutes to 2 hours, optimally said stirring time being 60 minutes.

The process according to the present invention comprises step c) of filtering the first suspension of step b), obtaining a first filtered solid comprising midostaurin.

Said filtration can be carried out using any of the methods known to those skilled in the art for this purpose, such as for example vacuum or centrifugal filtration, and paper, fabric or sintered glass as a filter medium. Preferably, said filtration is carried out under vacuum on sintered glass with a porosity of 5-15 Micron.

Preferably, said first filtered solid of step c) is washed with a further amount of water before step d) of the process according to the present invention.

Preferably for said washing with water before step d) a further amount of water is used varying from 10 to 30 milliliters per gram of said first filtrate of step c), more preferably varying from 15 to 25 milliliters per gram of said first filtrate of step c), optimally said further amount of water being 20 milliliters per gram of said first filtrate of step c).

Subsequently to said step c), the process according to the present invention comprises step d) of suspending under stirring the first filtered solid of step c) in a second amount of water so as to obtain a second suspension of midostaurin.

Preferably, in said step d) said second amount of water varies from 10 to 30 milliliters per gram of first filtered solid of step c), more preferably it varies from 15 to 25 milliliters per gram of said first filtered solid of step c), optimally said second amount of water being 20 milliliters per gram of said first filtered solid of step c).

Preferably, in said step d) of the process according to the present invention said stirring is maintained for a time varying from 30 minutes to 3 hours, more preferably varying from 45 minutes to 2 hours, optimally said stirring time being 60 minutes.

Preferably, step d) of the process according to the present invention is carried out at a temperature varying from 20° C. to 40° C., more preferably varying from 20° C. to 30° C., optimally said temperature being 25° C.

The process according to the present invention therefore comprises step e) of filtering the second suspension of step d), obtaining a second filtered solid comprising midostaurin.

Said filtration can be carried out using any of the methods known to those skilled in the art for this purpose, such as filtration under vacuum, and paper, fabric or sintered glass as a filter medium. Preferably, said filtration is carried out under vacuum on sintered glass with a porosity of 5-15 Micron.

In a preferred embodiment of the process according to the present invention, steps d) and e) are repeated at least once, more preferably at least twice, even more preferably at least three times, each time re-suspending the second filtrate in water according to the manners described for step d) and re-filtering it according to the manners described for step e) of the present process.

This leads to a further improvement of the process according to the present invention, in terms of residual content of organic solvent, which can thus advantageously reach extremely low values, of the order of hundreds of ppm.

Preferably, therefore, the process according to the present invention provides that said second filtered solid of step e) is re-suspended and subsequently refiltered according to said steps d) and step e) of the process, for at least three times.

The process according to the present invention also provides for step f) of drying the second filtered solid of step e), so as to obtain a dried solid, wherein said dried solid is said amorphous form of midostaurin.

Preferably, said step f) is carried out under vacuum, more preferably at a pressure varying from 1 to 50 mbar, even more preferably varying from 2 to 10 mbar, optimally said pressure being 5 mbar.

Preferably, said step f) is carried out at a temperature varying from 40° C. to 80° C., more preferably varying from 50° C. to 70° C., optimally said temperature being 60° C.

Preferably, said step f) is carried out for a time varying from 6 hours to 96 hours, more preferably varying from 16 to 24 hours.

Preferably, the amorphous form of midostaurin obtained from the process according to the present invention comprising a residual amount of dimethylsulfoxide, wherein said residual amount of dimethylsulfoxide is less than or equal to 5000 ppm.

In a preferred embodiment of the process, said residual amount of dimethylsulfoxide varies from 100 ppm to 5000 ppm, more preferably it varies from 150 ppm to 2500 ppm.

The process according to the present invention is therefore very simple, effective, industrially applicable, economically very convenient and allows to obtain an amorphous form of midostaurin with a low content of residual organic solvent, in such an amount so as to fall within the limits prescribed by the International Council for Harmonization of Technical Requirements for Pharmaceuticals for Human Use (ICH), which for the organic solvent used in the present process, dimethylsulfoxide, is 5000 ppm.

In a further aspect thereof, therefore, the present invention relates to an amorphous form of midostaurin comprising a residual amount of dimethylsulfoxide, wherein said residual amount of dimethylsulfoxide is less than or equal to 5000 ppm.

The Applicant has in fact found that said specific properties result from the process according to the present invention, from which the amorphous form of midostaurin according to the present invention is obtainable, and distinguish the latter from other known forms of midostaurin, also in the amorphous form.

In a preferred embodiment of said amorphous form of midostaurin, said residual amount of dimethylsulfoxide varies from 100 ppm to 5000 ppm, more preferably it varies from 150 ppm to 2500 ppm.

The determination of said residual amount of dimethylsulfoxide in the amorphous form of midostaurin according to the present invention can be advantageously carried out by means of headspace gas chromatography, by dissolving a sample of said amorphous form in N-methylpyrrolidone, and using an FID detector operating at a temperature of 250° C.

Thanks to the low residual amount of organic solvent, which falls within the limits prescribed by the International Council for Harmonization of Technical Requirements for Pharmaceuticals for Human Use (ICH), it is possible to use the amorphous form of midostaurin according to the present invention in the pharmaceutical field, and in particular for the treatment of tumors, in particular acute myeloid leukemia and mastocytosis, such as for example systemic mastocytosis, also in its aggressive systemic mastocytosis forms, systemic mastocytosis associated with a hematologic malignancy, and mastocytic leukemia.

In a further aspect, the present invention therefore relates to a pharmaceutical composition comprising the amorphous form of midostaurin according to the present invention, and at least one component selected from the group consisting of: a pharmaceutically acceptable excipient, and a pharmaceutically acceptable solvent.

Preferably, said pharmaceutical composition is in a form suitable for the enteral administration route, more preferably oral, and even more preferably in capsule or tablet form.

In a further aspect thereof, moreover, the present invention relates to the amorphous form of midostaurin according to the present invention or the pharmaceutical composition according to the present invention for use as a medicament.

In a further aspect thereof, the present invention further relates to the amorphous form of midostaurin according to the present invention or the pharmaceutical composition according to the present invention for use in the treatment of a tumor.

In a preferred embodiment, said tumor is acute myeloid leukemia or mastocytosis, more preferably a systemic mastocytosis, even more preferably a systemic mastocytosis selected from the group consisting of: aggressive systemic mastocytosis, systemic mastocytosis associated with a hematologic malignancy, and mastocytic leukemia.

### EXPERIMENTAL PART

The invention is now described by means of some examples to be considered for non-limiting illustrating purposes thereof.

### Examples

### Example 1

In a reactor with a volume of 5 liters and equipped with a thermo-adjustable jacket, 100 grams of midostaurin prepared according to the synthesis reported in the Italian patent application 102019000004729, were dissolved in 1000 milliliters of dimethylsulfoxide (DMSO). 1000 milliliters of water were added to the resulting solution, in 30 minutes maintaining the temperature at 25° C., observing the precipitation of the midostaurin previously in solution and thus obtaining a dispersion of midostaurin. After the end of the addition of water, the dispersion was maintained under stirring for 1 hour. Subsequently, the suspension was filtered under vacuum on sintered glass with porosity 5-15 Micron and the obtained filtered solid was washed with 2000 milliliters of water. The washed filtered solid was then subsequently loaded into a reactor in which 2000 milliliters of water were added under stirring, observing the formation of a further dispersion, which was maintained under stirring for 1 hour at a temperature of 25° C. Said suspension was then subsequently filtered under vacuum on sintered glass with porosity of 5-15 Micron. The obtained filtered solid was then dried under vacuum for 16 hours at 60° C., obtaining 95 grams of amorphous midostaurin.

The obtained amorphous form of midostaurin was then characterized to determine the residual amount of DMSO and by X-ray diffractometry.

The determination of the residual amount of DMSO was carried out by means of headspace gas chromatography, under the following conditions:
100 mg sample of the amorphous form dissolved in 1.0 ml of N-methylpyrrolidone;
DMSO standard 0.5 mg in 1.0 ml of N-methylpyrrolidone;
volume vial HSS 20 ml;
DB-624 column 30 m long, 0.53 mm internal diameter and 3 micron film;
carrier He with constant flow of 1.85 ml/min;
injector temperature 250° C.;
FID detector operating at a temperature of 250° C.;
vial equilibration at 100° C. for 15 minutes with the following temperature schedule:

| time | Temperature | Heating rate | |
|---|---|---|---|
| (minutes) | (° C.) | (° C./minute) | |
| 0-5 | 40 | — | Isothermal condition |
| 5-30 | 40 → 90 | 2 | Linear thermal gradient |
| 30-35.3 | 90 → 250 | 30 | Linear thermal gradient |
| 35.3-50.3 | 250 | — | Isothermal condition |

The analysis showed a residual amount of DMSO equal to 2000 ppm.

The characterization by X-ray diffractometry was done using an X-ray diffractometer (operating with a voltage of 45 kV, current of 40 mA, scan duration 30 minutes, CuKα source angle range 2θ from 3° to 50°).

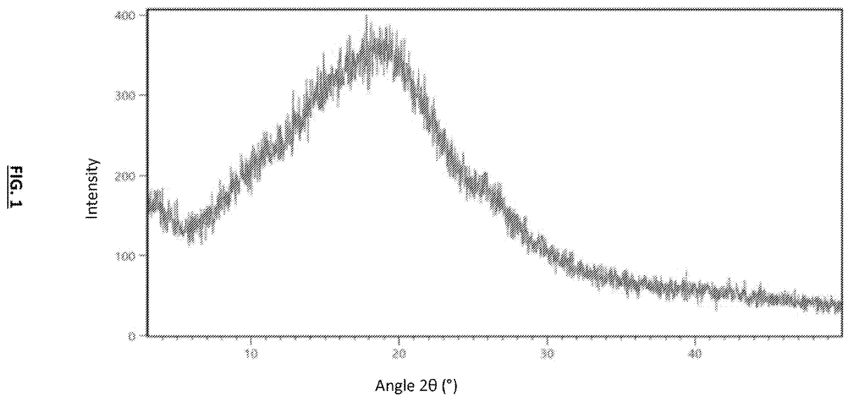

FIG. 1 shows the X-ray diffractogram obtained.

### Example 2

In a reactor with a volume of 5 liters and equipped with a thermo-adjustable jacket, 100 grams of midostaurin prepared according to the synthesis reported in the Italian patent application 102019000004729, were dissolved in 1000 milliliters of dimethylsulfoxide (DMSO). 1000 milliliters of water were added to the resulting solution, in 30 minutes maintaining the temperature at 25° C., observing the precipitation of the midostaurin previously in solution and thus obtaining a dispersion of midostaurin. After the end of the addition of water, the dispersion was maintained under stirring for 1 hour. Subsequently, the suspension was filtered under vacuum on sintered glass with porosity 5-15 Micron and the obtained filtered solid was washed with 2000 milliliters of water. The washed filtered solid was then subsequently loaded into a reactor in which 2000 milliliters of water were added under stirring, observing the formation of a further dispersion, which was maintained under stirring for 1 hour at a temperature of 25° C. Said suspension was then subsequently filtered under vacuum on sintered glass with porosity 5-15 Micron and this operation of dispersion in water and filtration was repeated three times. The filtered solid obtained after said three repetitions was then dried under vacuum for 16 hours at 60° C., obtaining 95 grams of amorphous midostaurin.

The obtained amorphous form of midostaurin was then characterized to determine the residual amount of DMSO and by X-ray diffractometry.

The obtained amorphous form of midostaurin was then characterized to determine the residual amount of DMSO and by x-ray diffractometry, as described in Example 1.

The analysis of the residual amount of DMSO showed a residual amount of the latter equal to 200 ppm.

The x-ray diffractogram of the amorphous form of midostaurin according to Example 2 was found to be completely similar to that obtained in Example 1.

### Example 3

In a reactor with a volume of 2 liters, 100 grams of midostaurin, prepared according to the synthesis reported in Italian patent application 102019000004729, were dissolved in 1000 milliliters of dimethylsulfoxide (DMSO). The resulting solution was then added to a second reactor with a volume of 5000 milliliters and equipped with a thermo-adjustable jacket, containing 1000 milliliters of water, in 30 minutes maintaining the temperature at 25° C., observing the precipitation of the midostaurin previously in solution and thus obtaining a dispersion of midostaurin. After the end of the addition of water, the dispersion was maintained under stirring for 1 hour. Subsequently, the suspension was filtered under vacuum on sintered glass with a porosity of 5-15 Micron and the obtained filtered solid was washed with 2000 milliliters of water. The washed filtered solid was then subsequently loaded into a reactor in which 2000 milliliters of water were added under stirring, observing the formation of a further dispersion, which was maintained under stirring for 1 hour at a temperature of 25° C. Said suspension was then subsequently filtered under vacuum on sintered glass with porosity 5-15 Micron and this operation of dispersion in water and filtration was repeated three times. The filtered solid obtained after said three repetitions was then dried under vacuum for 16 hours at 60° C., obtaining 95 grams of amorphous midostaurin.

The obtained amorphous form of midostaurin was then characterized to determine the residual amount of DMSO and by x-ray diffractometry, as described in Example 1.

The analysis of the residual amount of DMSO showed a residual amount of the latter equal to 300 ppm.

The x-ray diffractogram of the amorphous form of midostaurin according to Example 3 was found to be completely similar to that obtained in Example 1.

The tests carried out have made it possible to highlight how the process according to the present invention allows to obtain an amorphous form of midostaurin with a content of residual organic solvent, DMSO, falling within the limits prescribed by the International Council for Harmonization of Technical Requirements for Pharmaceuticals. for Human Use (ICH) and therefore compatible with use in the pharmaceutical field.

1. A process for the preparation of an amorphous form of midostaurin, comprising the steps of:
a) preparing a solution of midostaurin in dimethylsulfoxide;
b) combining the solution of step a) with a first amount of water so as to obtain a first suspension of midostaurin;
c) filtering the first suspension of step b), obtaining a first filtered solid comprising midostaurin;
d) suspending under stirring the first filtered solid of step c) in a second amount of water so as to obtain a second suspension of midostaurin;
e) filtering the second suspension of step d), obtaining a second filtered solid comprising midostaurin; and
f) drying the second filtered solid of step e) so as to obtain a dried solid, wherein said dried solid is said amorphous form of midostaurin.

2. The process according to claim 1, wherein in said solution of step a) the amount of dimethylsulfoxide varies from 1 to 30 milliliters per gram of midostaurin.

3. The process according to claim 1, wherein said first amount of water varies from 1 to 30 milliliters per gram of midostaurin in the solution of step a).

4. The process according to claim 1, wherein said first amount of water varies from 0.5 to 1.5 parts in volume per part in volume of dimethylsufoxide of the solution of step a).

5. The process according to claim 1, wherein said step b) is carried out at a temperature varying from 20° C. to 40° C.

6. The process according to any one of claims from 1 to 5, wherein in said step b) said solution of step a) is added to said first amount of water in a time varying from 5 minutes to 3 hours.

7. The process according to claim 1, wherein in said step b) said first amount of water is added to said solution of step a) in a time varying from 5 minutes to 3 hours.

8. The process according to claim 1, wherein said first filtered solid comprising midostaurin of step c) is washed with water before step d).

9. The process according to claim 1, wherein in said step d) said second amount of water varies from 10 to 30 milliliters per gram of first filtrate of step c).

10. The process according to claim 1, wherein in said step d) said stirring is maintained for a time varying from 30 minutes to 3 hours.

11. The process according to claim 1, wherein said second filtered solid of step e) is resuspended and successively refiltered according to said steps d) and e), at least once.

12. The process according to claim 1, wherein said step f) is carried out under vacuum.

13. The process according to claim 1, wherein said step f) is carried out at a temperature varying from 40° C. to 80° C.

14. The process according to claim 1, wherein said step f) is carried out for a time varying from 6 hours to 96 hours.

15. The process according to claim 1, wherein said amorphous form of midostaurin comprises a residual amount of dimethylsulfoxide, wherein said residual amount is less than or equal to 5000 ppm.

16. The process according to claim 15, wherein said residual amount of dimethylsulfoxide varies from 100 to 5000 ppm.

17. An amorphous form of midostaurin comprising a residual amount of dimethylsulfoxide, wherein said residual amount is less than or equal to 5000 ppm.

18. The amorphous form of midostaurin according to claim 17, wherein said residual amount of dimethylsulfoxide varies from 100 to 5000 ppm.

19. A pharmaceutical composition comprising:
an amorphous form of midostaurin obtainable by means of the process according to claim 1 or comprising a residual amount of dimethylsulfoxide less than or equal to 5000 ppm, and
at least one component selected from the group consisting of: a pharmaceutically acceptable excipient, and a pharmaceutically acceptable solvent.

20. (canceled)

21. A method for treating a tumor in a patient in need thereof, said method comprising administering to said patient
an amorphous form of midostaurin obtainable by means of the process according to claim 1; or
an amorphous form of midostaurin comprising a residual amount of dimethylsulfoxide less than or equal to 5000 ppm and optionally at least one component selected from the group consisting of a pharmaceutically acceptable excipient and a pharmaceutically acceptable solvent.

22. The method amorphous according to claim 21, wherein said tumor is acute myeloid leukemia or a mastocytosis.

23. The method amorphous according to claim 22, wherein said mastocytosis is a systemic mastocytosis.

24. The method according to claim 23, wherein said systemic mastocytosis is selected from the group consisting of: aggressive systemic mastocytosis, systemic mastocytosis associated with a hematologic malignancy, and mastocytic leukemia.
